(19) United States
(12) Patent Application Publication
(10) Pub. No.: US 2009/0216264 A1
(43) Pub. Date: Aug. 27, 2009

(54) IMPLANTABLE CLOSURE APPARATUS AND METHODS

(76) Inventors: Paul A. Friedman, Rochester, MN (US); Samuel J. Asirvatham, Rochester, MN (US); Charles J. Bruce, Rochester, MN (US)

Correspondence Address: MUETING, RAASCH & GEBHARDT, P.A., P.O. BOX 581336, MINNEAPOLIS, MN, 55458-1336, US

(21) Appl. No.: 12/067,373

(22) PCT Filed: Sep. 19, 2006
(86) PCT No.: PCT/US06/36468

§ 371 Date: Sep. 10, 2008

Related U.S. Application Data
(60) Provisional application No. 60/718,562, filed on Sep. 19, 2005.
(60) Provisional application No. 60/778,224, filed on Mar. 2, 2006.

(51) Int. Cl. A61B 17/08

(52) U.S. Cl. 606/213

(57) ABSTRACT

Implantable closure apparatus and methods for sealing or closing openings at internal body locations. The apparatus and methods may involve the delivery and attachment of a patch, patch and plug, or plug only to seal or close the opening. The closure apparatus and methods may be used to close a patent foramen ovale (PFO).

### RELATED APPLICATIONS

This application claims the benefit under 35 U.S.C. § 119(e) of U.S. Provisional Patent Application Ser. No. 60/718,562, entitled IMPLANTABLE CLOSURE APPARATUS AND METHODS, filed Sep. 19, 2005 (Attorney Docket No. 230.00280160) and of U.S. Provisional Patent Application Ser. No. 60/778,224, entitled IMPLANTABLE CLOSURE APPARATUS AND METHODS, filed Mar. 2, 2006 (Attorney Docket No. 230.00320160), both of which are hereby incorporated by reference in their respective entireties.

The present invention relates to the field of implantable closure apparatus. More particularly, the present invention relates to implantable closure apparatus that are adapted to, e.g., close a patent foramen ovale in a heart.

As discussed in U.S. Patent Application Publication No. US 2005/0034735 (Deem et al.), the presence of a patent foramen ovale (PFO) may be associated with an increased risk of cryptogenic stroke and migraines. Of the many different devices and techniques developed to address conditions such as PFO's, a number of disadvantages may be seen. For example, the devices are often bully, which can cause thrombosis or inadvertent distortion/compression of important adjacent structures. Many, if not all, of the devices have one or more components that remain in the left atrium after deployment where, if thrombosis occurs, there is a chance of stroke. Further, the devices can typically close only gross defects, not multiple smaller perforations (which often exist).

Other potential disadvantages include, e.g., that during deployment some of these devices intentionally perforate the atrial septum which may cause additional problems. Many of the devices are also difficult to place, potentially resulting in “oblique positioning” and subsequent leakage. The devices often prevent subsequent procedures from being performed if the subsequent procedures require crossing the atrial septum. In addition, many of these devices are “one-shot” deployments. That is, there is only limited ability for positioning the device after it has been deployed. Further, many of the devices will only work if the separated overlapping remnants of the septum primum and the septum secondum that form the overlapping margins of the PFO are intact and/or large enough to contact each other.

### SUMMARY OF THE INVENTION

The present invention provides implantable closure apparatus and methods for sealing or closing openings at internal body locations. The apparatus and methods may preferably involve the delivery and attachment of a patch, patch and plug, or plug only to seal or close the opening.

The closure apparatus and methods of the present invention may provide a variety of advantages. Among the potential advantages is the ability to close a PFO using apparatus and devices inserted into and residing only in the right atrium (preferably percutaneously), preferably avoiding foreign body placement in the left atrium (which may increase stroke risk). Another potential advantage is that the apparatus and method preferably do not create any additional holes or perforations in the atrial septum during deployment. A further potential advantage is that the apparatus itself does not cross or span the atrial septum (through a PFO or other opening).

Other potential advantages of the closure apparatus and methods of the present invention may include no, or minimal, foreign bodies remaining in a subject's body after deployment. More specifically, the apparatus and methods of the present invention preferably do not involve leaving any foreign bodies in the left atrium (and in many instances, may not even require any access or entry into the left atrium (even temporarily)).

Still another potential advantage of the closure apparatus and methods of the present invention is that the ability to transect the atrial septum in subsequent procedures may be retained. This is typically difficult or impossible with current bulky devices used to close PFO's. In some instances, the closure apparatus may be re-positioned, re-deployed, or be deployed multiple times in different areas if required.

Yet another potential advantage of the closure apparatus and methods of the present invention is that a relatively large PFO or multiple, smaller perforations in a localized area may be closed. In addition, at least some of the closure apparatus and methods of the present invention may be used to treat PFO's where the septum primum and septum secondum are not able to be apposed to one another (unlike some known devices).

Although the present invention is described herein in connection with the closure of PFO's, it should be understood that the closure apparatus and methods may find use in a variety of other internal body locations in which openings are to be sealed or closed. These may include larger defects (ASD, VSD, etc.) and smaller ones (e.g., vessel perforations, etc.).

In some embodiments, the closure apparatus described herein involve the use of a patch to cover a PFO or other opening. The patches used may be in a variety of different forms. It may be preferred that the patches be collapsible such that they can be delivered to an internal body site through, e.g., a lumen in a sheath. The patches may be provided in any suitable shape, although those depicted herein are circular (which may be preferred).

Some of the patches may be constructed in the form of a synthetic or natural membrane. Some potentially suitable natural materials may include, e.g., porcine pericardium, human pericardium, albumin, collagen, fibrin-based membranes, etc. Some potentially suitable synthetic membrane materials may include, e.g., cyanoacrylates, polytetrafluoroethylene, etc.

Still other patches may be provided in the form of a porous or mesh body that may be designed to promote cell ingrowth after implantation. Some potentially suitable constructions may include, e.g., non-woven materials, woven materials, knitted materials, metallic (or other) matrices, etc. Porous patches may be provided in combination with materials that promote cellular ingrowth, e.g., cell recruitment factors (VEGF, EGF, FGF, PDGF, etc.).

Other patches of the present invention may be constructed of degradable materials such that, over time, the amount of patch material at the deployment site would be reduced (e.g., it may be replaced by tissue). For example, the patch could be constructed of a degradable bio-polymer.

It may be preferred that the patches used in some embodiments of the present invention be secured at the internal body site by “tissue welding” via radio-frequency (RF) energy, thermal energy, etc. The energy could be supplied via the scaffolds used to deploy the patch or by a second device (e.g., a catheter, etc.). Various solders made of albumin, collagen, fibrin, etc. may be provided on or in the patch to assist in attachment of the patch to existing tissue as discussed in at least some of the documents identified herein. In some instances, the patch itself may be used to conduct energy to perform tissue welding over all or substantially all of the surface of the patch. For example, the patch may include electrically conductive elements and/or coatings suitable to transmit energy.

In some apparatus and methods of the present invention, a device may be used to attach itself to the tissue at the opening to be closed (e.g., the septum primum, septum secundum, etc. in the case of PFO) and bring the tissues together for fastening by any technique (e.g., RF welding, adhesives, sutures, clips, patches, etc.). The device may use cryogenic energy to attach itself to the tissue (e.g., septum primum, septum secundum, etc. in the left or right atrium in the case of a PFO). The cryogenic device could be used to at least temporarily hold the tissues in selected positions during, e.g., attachment of the patch, plug or other closure element. The cryogenic tissue apposition may be accomplished using, e.g., a cryoprobe or other device. In some instances, the same device may be used to co-apt opposing tissue and attach the patch or weld tissue as discussed herein. One potentially suitable device for delivering RF energy in combination with cryogenic energy may be described in U.S. Patent Application Publication No. US 2004/0116921 (Sherman et al.).

In one aspect, the present invention may provide a percutaneous closure apparatus that includes a delivery sheath with a proximal end and a distal end, wherein one or more lumens extend from the proximal end to the distal end of the delivery sheath; a collapsible patch having first and second major surfaces, wherein the patch is capable of collapsing into a delivery configuration amenable for delivery to an internal body location through one lumen of the one or more lumens of the delivery sheath, and wherein the patch is capable of moving from the delivery configuration into a deployment configuration in which the first and second major surfaces are generally flat when the patch is located outside of the one lumen of the one or more lumens; a deployment scaffold attached to the patch, wherein the deployment scaffold is capable of collapsing into a delivery configuration amenable for delivery to an internal body location through the one lumen of the one or more lumens of the delivery sheath, and wherein the deployment scaffold is capable of moving from the delivery configuration into a deployment configuration in which the deployment scaffold supports the patch in its deployment configuration when the deployment scaffold and the patch are located outside of the one lumen of the one or more lumens; and a plurality of struts attached to the deployment scaffold, wherein each strut extends from the deployment scaffold to the proximal end of the delivery sheath, and wherein the plurality of struts can move proximally and distally within the one lumen of the one or more lumens of the delivery sheath to move the patch and deployment scaffold within the delivery sheath.

In another aspect, the present invention may provide a method of closing a patent foramen ovale (PFO) by positioning a distal end of a delivery sheath into the right atrium of a subject; advancing a closure apparatus out of a lumen of the delivery sheath and into the right atrium, wherein the closure apparatus does not enter the left atrium. The closure apparatus may include a collapsible patch having first and second major surfaces, wherein the patch is capable of collapsing into a delivery configuration amenable for delivery to an internal body location through one lumen of the one or more lumens of the delivery sheath, and wherein the patch is capable of moving from the delivery configuration into a deployment configuration in which the first and second major surfaces are generally flat when the patch is located outside of the one lumen of the one or more lumens; a deployment scaffold attached to the patch, wherein the deployment scaffold is capable of collapsing into a delivery configuration amenable for delivery to an internal body location through the one lumen of the one or more lumens of the delivery sheath, and wherein the deployment scaffold is capable of moving from the delivery configuration into a deployment configuration in which the deployment scaffold supports the patch in its deployment configuration when the deployment scaffold and the patch are located outside of the one lumen of the one or more lumens; and a plurality of struts attached to the deployment scaffold, wherein each strut extends from the deployment scaffold to the proximal end of the delivery sheath, and wherein the plurality of struts can move proximally and distally within the one lumen of the one or more lumens of the delivery sheath to move the patch and deployment scaffold within the delivery sheath. The method may further include positioning a second major surface of the patch over one or more perforations in the atrial septum, wherein blood flow through the one or more perforations is inhibited by the patch; and attaching the patch to the atrial septum by directing RF energy towards the atrial septum through a first major surface of the patch, wherein the RF energy passes through the second major surface of the patch and into the atrial septum, wherein the attaching includes directing the RF energy to a series of discrete locations on the patch.

In another aspect, the present invention may provide a method of closing a patent foramen ovale in an atrial septum by positioning a distal end of a first delivery sheath in the right atrium of a subject; positioning a distal end of a second delivery sheath in the left atrium of a subject: advancing a first component out of a lumen of the first delivery sheath and into the right atrium, wherein the first component is located on one side of the atrial septum; advancing a second component out of a lumen of the second delivery sheath and into the left atrium, wherein the second component is located opposite the first component on an opposing side of the atrial septum; magnetically aligning the first component and the second component across the atrial septum; delivering RF energy to the patent foramen ovale in the atrial septum to close the patent foramen ovale; removing the second component and the second delivery sheath from the left atrium; and removing the first component and the first delivery sheath from the right atrium.

In another aspect, the present invention provides a percutaneous closure apparatus that optionally includes a delivery sheath having a proximal end and a distal end, wherein one or more lumens extend from the proximal end to the distal end of the delivery sheath; a collapsible patch with first and second major surfaces, wherein the patch is capable of collapsing into a delivery configuration amenable for delivery to an internal body location through one lumen of the one or more lumens of the delivery sheath, and wherein the patch is capable of moving from the delivery configuration into a deployment configuration in which the first and second major surfaces are generally flat when the patch is located outside of the one lumen of the one or more lumens. The closure apparatus may optionally include a deployment scaffold attached to the patch, wherein the deployment scaffold is capable of collapsing into a delivery configuration amenable for delivery to an internal body location through the one lumen of the one or more lumens of the delivery sheath, and wherein the deployment scaffold is capable of moving from the delivery configuration into a deployment configuration in which the deployment scaffold supports the patch in its deployment configuration when the deployment scaffold and the patch are located outside of the one lumen of the one or more lumens; Another feature that may be included in the closure apparatus is a clamping arm having a first end attached to the deployment scaffold, the clamping arm including a resilient curved member that generates a spring force when deformed that is sufficient to retain the collapsible patch in a selected location. A tissue anchor may be attached at a second end of the clamping arm, wherein the second end of the clamping arm can be secured to a selected internal location. The closure apparatus may also include an advancement member extending through the delivery sheath, the advancement member attached to the clamping arm by a connector, wherein the connector provides one or more degrees of freedom in movement between the advancement member and the clamping arm.

In another aspect, the present invention may include a method of closing a patent foramen ovale by providing a closure apparatus that includes a clamping arm as discussed herein; positioning the distal end of the delivery sheath into the right atrium of a subject; advancing the closure apparatus out of a lumen of the delivery sheath and into the right atrium; and positioning the collapsible patch at a selected location over one or more perforations in the atrial septum, wherein blood flow through the one or more perforations is inhibited by the patch. The collapsible patch is (at least temporarily) retained at the selected location by advancing the second end of the clamping arm into the coronary sinus and attaching the tissue anchor to tissue in the coronary sinus, wherein the clamping arm is partially deformed such that a spring force is generated between the first end and the second end of the clamping arm. Optionally, the patch may be attached to the tissue surrounding the PFO using any suitable technique as discussed herein, e.g., by directing RF energy towards the atrial septum towards the collapsible patch, wherein the RF energy passes through the patch and into the atrial septum, wherein the RF energy welds the patch to the atrial septum.

These and other features and advantages of the present invention may be described in connection with the exemplary embodiments of the invention below.

### BRIEF DESCRIPTIONS OF THE FIGURES

FIG. 1 is a perspective view of the distal end of a delivery sheath that may be used in connection with the apparatus of the present invention.

FIG. 2 is a perspective view of the delivery sheath of FIG. 1 with a collapsible patch in a partially collapsed configuration being deployed out of the delivery sheath.

FIG. 3 is a perspective view of the apparatus of FIGS. 1-2, with the patch deployed and supported by a deployment scaffold in its deployment configuration.

FIGS. 4 & 5 are perspective views of the apparatus of FIG. 3, in which the deployed patch is depicted as canted relative to the delivery sheath.

FIGS. 6 & 7 depict an optional feature of the apparatus of FIGS. 1-5 in the form of a removable connection between the perimeter support and the struts in an apparatus according to the present invention.

FIGS. 8-10 depict another exemplary embodiment of a removable connection that can be made between a perimeter support and strut in an apparatus according to the present invention.

FIGS. 11 A & 11B depict an exemplary perimeter support in combination with a pair of cross-members, with a releasable connection made to the cross-members.

FIG. 12 depicts advancement of a delivery sheath into a right atrium where a patent foramen ovale is located.

FIG. 13 depicts an exemplary closure apparatus extending out of the delivery sheath of FIG. 12.

FIG. 14 depicts one method of attaching the patch of FIG. 13 to the atrial septum within the right atrium.

FIG. 15 depicts one shaded area of the patch of FIG. 14 that is already attached to the atrial septum.

FIGS. 16 & 17 depict attachment of the perimeter of the patch of FIG. 15 to the atrial septum.

FIG. 18 depicts the patch of FIG. 17 attached to the atrial septum after withdrawal of the delivery sheath from the right atrium.

FIG. 19 is a perspective view of another exemplary closure apparatus that includes an optional delivery sheath with a patch deployed from the distal end of the delivery sheath, wherein the patch includes a line of weakness formed therein.

FIG. 20 depicts the deployment of a secondary scaffold from the delivery sheath in connection with the patch of FIG. 19.

FIG. 21 depicts the secondary scaffold of FIG. 20 after advancement out of the delivery sheath, with the secondary scaffold expanded to a deployment configuration.

FIG. 22 depicts the patch of FIG. 21 after separation along the line of weakness in the patch, with a portion of the patch located outside of the line of weakness being removed along with the perimeter support.

FIG. 23 depicts removal of the secondary scaffold in connection with removal of the perimeter support from the patch of FIG. 22.

FIG. 24 depicts another exemplary closure apparatus and method in which a first delivery sheath is deployed into the right atrium and a second delivery sheath is deployed into the left atrium using a retrograde aortic approach.

FIG. 25 depicts the delivery sheaths of FIG. 24 in position in the right and left atriums, with a first component deployed into the right atrium and a second component deployed into the left atrium.

FIG. 26 depicts the two components of FIG. 25 in alignment across the opening components and preferably advanced to compress the tissue located between them to preferably seal the defect.

FIG. 27 depicts another exemplary closure apparatus including a patch and a clamping arm in the form of a resilient curved member attached thereto.

FIG. 28 depicts another exemplary closure apparatus with a clamping arm that includes a coil and a plug attached to the patch.

FIG. 29 depicts the closure apparatus of FIG. 27 after advancement out of a delivery sheath.

FIG. 30 depicts one embodiment of a closure apparatus according to FIGS. 27-29 as deployed within a patient to close a PFO.

### EXEMPLARY EMBODIMENTS OF THE INVENTION

In the following description of some exemplary embodiments of the invention, reference is made to the accompanying figures which form a part hereof, and in which are shown, by way of illustration, specific embodiments in which the invention may be practiced. It is to be understood that other embodiments may be utilized and structural changes may be made without departing from the scope of the present invention.

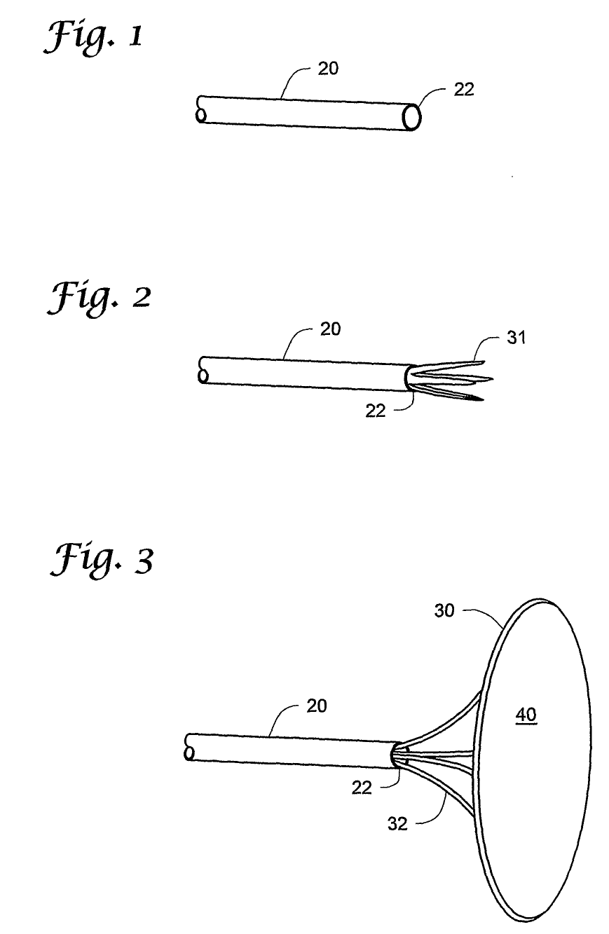

FIG. 1 is a perspective view of the distal end 22 of an optional delivery sheath 20 that may be used in the apparatus of the present invention. It is preferably in the form of a low profile, flexible tube that can be advanced to the heart via the vasculature over a wire using techniques and devices well known in the art. FIG. 2 is a perspective view of the delivery sheath of FIG. 1 in which a collapsible patch is being deployed out of the distal end 22 with the support of a deployment scaffold 31 that is depicted in its partially collapsed configuration.

FIG. 3 is a perspective view of the apparatus of FIGS. 1-2, with the patch 40 deployed and supported by the deployment scaffold 31 in its deployment configuration. The deployment scaffold 31 (in the depicted embodiment) includes a perimeter support 30 that is supported by struts 32 attached thereto, the struts extending through the lumen of the delivery sheath 20. It should be understood that the perimeter support 30 is optional. For example, some patches 40 may exhibit structural characteristics that may make the perimeter support 30 unnecessary. In such an embodiment, the struts 32 may be attached directly to the patch 40.

It may be preferred that at least some portions of the deployment scaffold be constructed of, e.g., shape memory materials to assist in the changes between the delivery configuration and the deployment configuration. For example, the perimeter support 30 may be constructed of shape memory metals, polymers, etc.

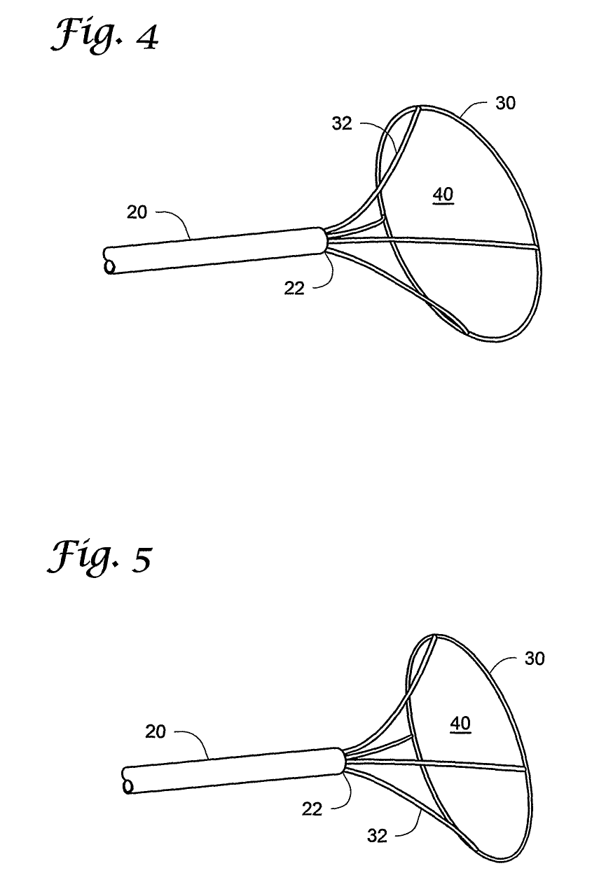

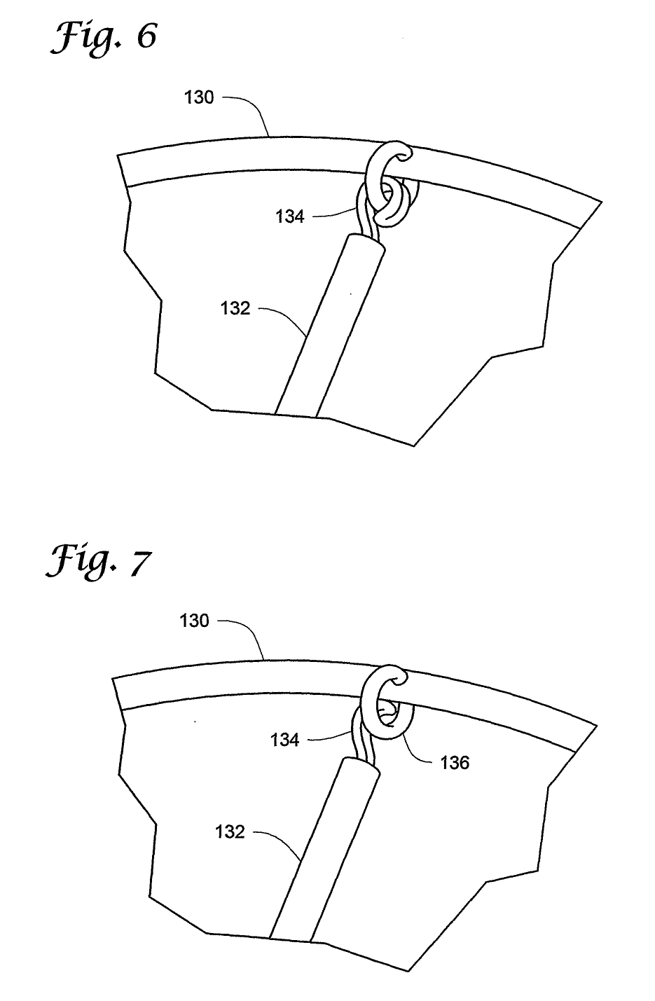

FIGS. 4 & 5 are perspective views of the apparatus of FIG. 3, in which the deployed patch 40 is depicted as canted relative to the delivery sheath 20 (as opposed to being oriented perpendicular to a longitudinal axis extending out of the lumen of the delivery sheath 20). Canting of the patch 40 (and associated deployment scaffold 30) may preferably be accomplished by selectively manipulating the different struts 32 from the proximal end of the delivery sheath 20. Canting and tilting may facilitate tissue contact given that the fossa ovalis may not be orthogonal to the sheath at the time of deployment. Additional control may be added by use of a steerable sheath (not shown). This control may allow for better approximation of the patch 40 based on, e.g., local patient-specific anatomy.

FIGS. 6 & 7 depict an optional feature of the apparatus of FIGS. 1-5. Although the struts 32 may be permanently attached to the perimeter support 30, it may be preferred that the struts be removably attached. The embodiment depicted in FIGS. 6 & 7 is designed to achieve that by providing struts 132 that are removably attached to the perimeter support 130 of a deployment scaffold. The attachment is made through an interlocking mechanical connection such as, e.g., the hook 134 and loop 136 structure depicted in FIGS. 6 & 7.

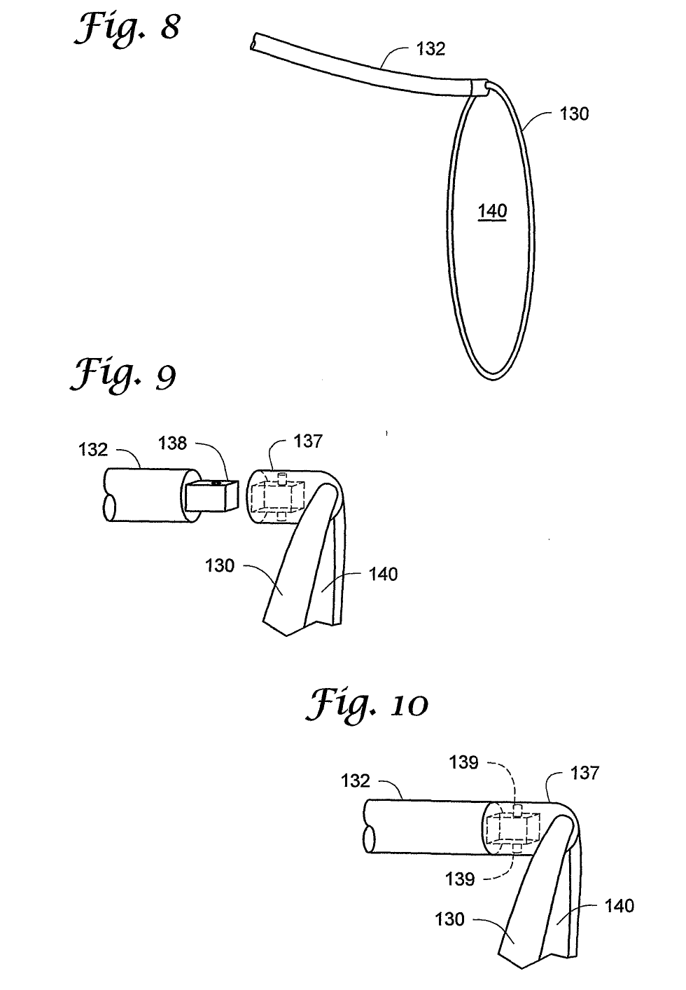

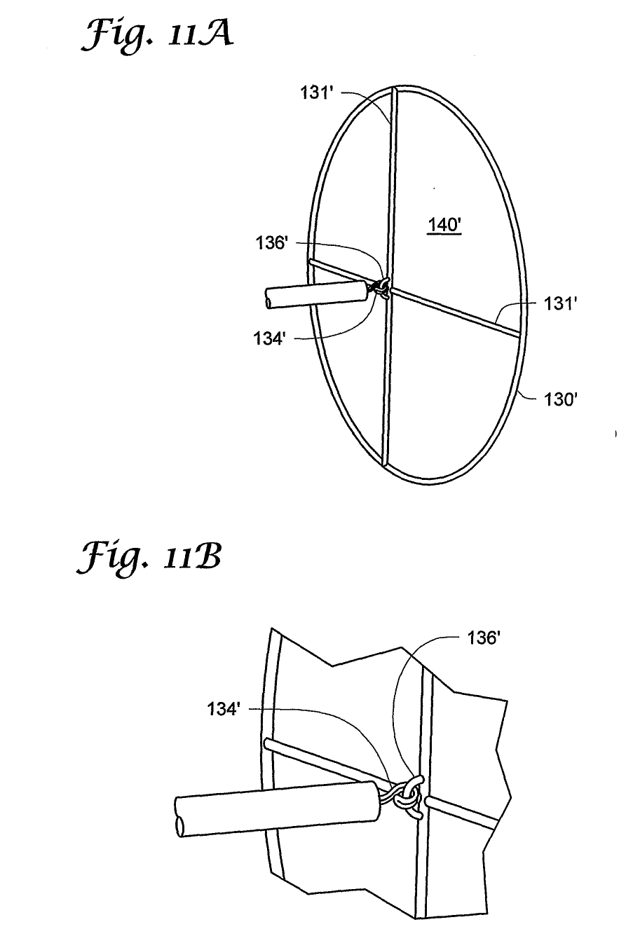

Another exemplary embodiment of a removable connection that can be made between a perimeter support 130 and strut 132 is depicted in FIGS. 8-10. As depicted, the perimeter support 130 may include a socket 137 adapted to receive a complementary fitting 138 located at the end of the strut 132. The socket 137 and fitting 138 may preferably exhibit magnetic attraction towards each other (using permanent magnets, electromagnets, etc.) to assist in connecting the components. As seen in FIG. 10, the fitting 138 may include retractable pins 139 that are received in complementary voids in the socket 137 to secure the fitting 138 to the socket 137 after the fitting 138 is in position within the socket 137.

The releasable connection depicted in FIGS. 8-10 may be used in initial deployment of the patch 140 and/or connections like those depicted may be used to reposition, retrieve and/or redeploy the patch 140 after an initial deployment. Although only one such connection is depicted on one strut in FIGS. 8-10, it should be understood that the connections could be provided at the ends of more than one strut. Furthermore, such a connection may be provided but not used in the initial deployment of the patch 140, with the connection being used for repositioning, retrieval, and/or redeployment after an initial deployment.

Although the releasable connections depicted in FIGS. 6-10 are attached to the perimeter support 130 of the patch 140, the deployment scaffold may include additional support members that span the patch 140. One exemplary embodiment of such a deployment scaffold is depicted in FIGS. 11A & 11B in which a perimeter support 130′ is provided in combination with a pair of cross-members 131′ spanning the patch 140′. Although not required, the cross-members 131′ intersect proximate the center of the patch 140′. In addition to providing additional support for the deployment scaffold, the cross-members may also provide additional locations for releasable connectors that may be used for initial deployment, positioning, repositioning, retrieval, and/or redeployment of the patch 140′. One such releasable connection is depicted in FIGS. 11A & 11B in the form of a hook 134′ and loop 136′ similar to those depicted in FIGS. 6 & 7.

Although two exemplary releasable connectors are depicted in FIGS. 6-10, it should be understood that the apparatus of the present invention may use a wide variety of connectors using any suitable connection technology or combination of two or more technologies. Examples may include mechanical connection, magnetic connection, adhesive connection, etc.

FIGS. 12-18 depict one method of using an apparatus according to the present invention to close a patent foramen ovale or other opening in the atrial septum between the left and right atria of the heart.

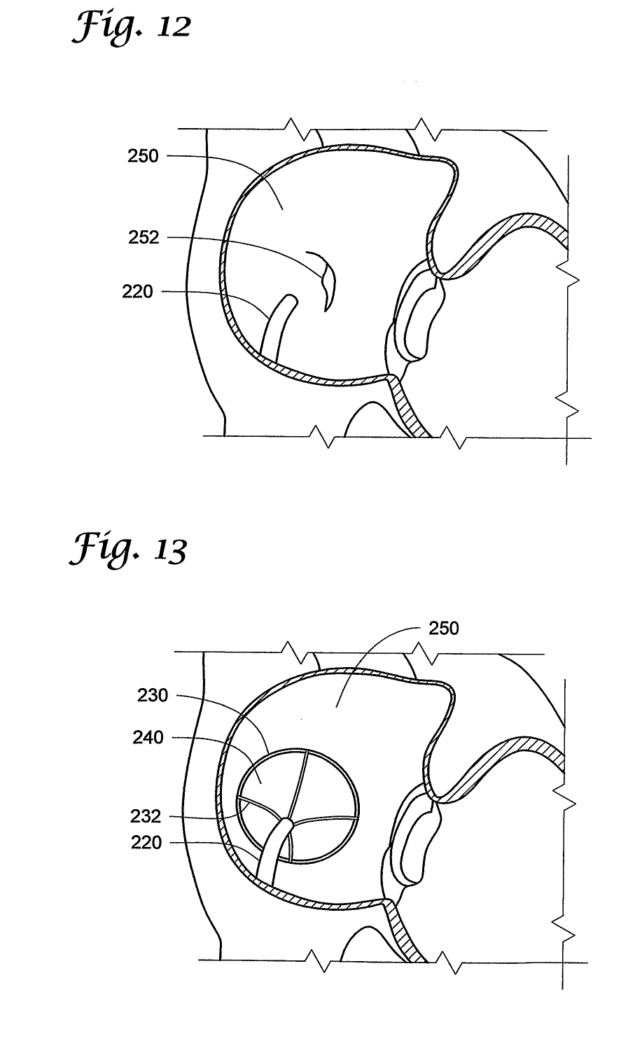

FIG. 12 depicts advancement of a delivery sheath 220 into the right atrium 250 where a patent foramen ovale 252 is located. FIG. 13 depicts a closure apparatus extending out of the delivery sheath 220. The apparatus includes a deployment scaffold in the form of a perimeter support 230 and struts 232 used to support a patch 240 within the right atrium. Manipulation of the delivery sheath 220 and/or struts 232 preferably enables a practitioner to position the patch 240 so that one of its major surfaces is located over the opening 252.

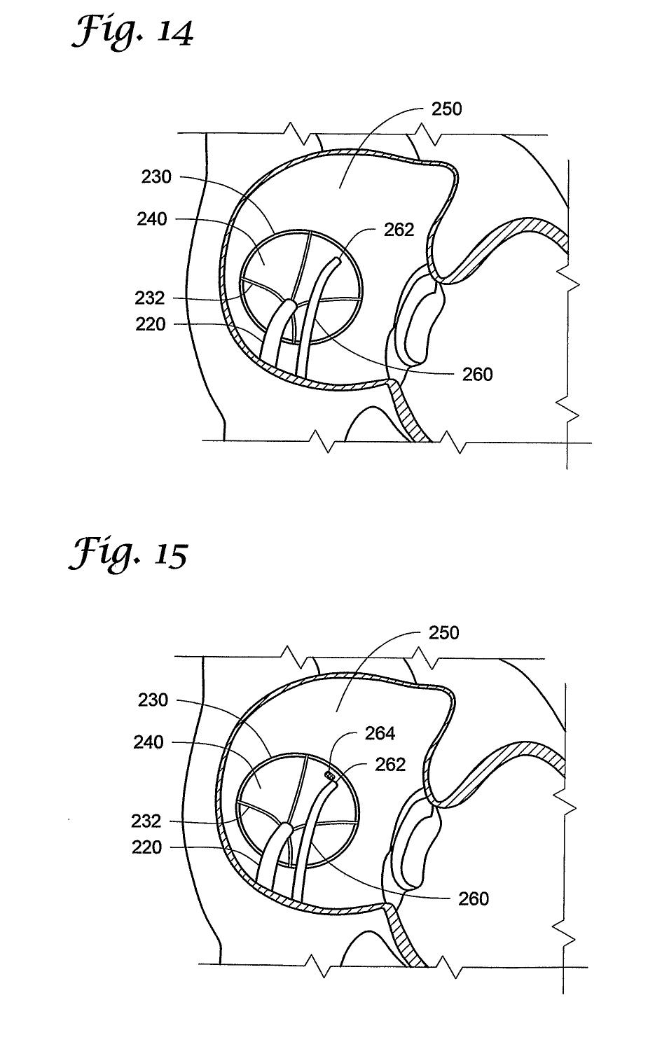

FIG. 14 depicts one method of attaching the patch 240 to the atrial septum within the right atrium 250. With the patch 240 in place, an attachment catheter 260 may preferably be advanced into the right atrium 250. Although depicted as a separate device delivered independently of the delivery sheath 220, the attachment catheter 260 could potentially be advanced through a second lumen in the delivery sheath 220 if both devices were appropriately sized.

The attachment catheter 260 is preferably capable of delivering energy (e.g., optical, thermal, RF, etc.) through its distal end 262 to the patch 240 sufficient to attach the patch 240 to the atrial septum. One area 264 of the patch 240 that is already attached is depicted as shaded in FIG. 15.

Methods of using energy (e.g., RF, thermal, etc.) to perform “tissue welding” that may be suitable for use in attaching patches in accordance with the present invention may be described in, e.g., International Publication No. WO 2004/086944 (Malecki et al.) and U.S. Patent Application Publication No. U.S. 2005/0034735 (Deem et al.), as well as U.S. Pat. No. 6, 391,049 (McNally et. al.); U.S. Pat. No. 5,156,613 (Sawyer); U.S. Pat. No. 5,669, 934 (Sawyer); U.S. Pat. No. 5,824,015 (Sawyer); and U.S. Pat. No. 5,931,165 (Reich et al.). These technologies all disclose the use of energy delivery to tissue solders and patches in order to join tissue and form anastamoses between arteries, bowel, nerves, etc. Also of interest may be laser suturing of biological materials (e.g., U.S. Pat. Nos. 5,725,522; 5,569,239; 5,540,677; and 5,071,417 (all to Sinofsky). Other references, such as International Publication No. WO 03/053493 (Ryan et al.) describe devices for closing PFO's involving bioresorbable materials.

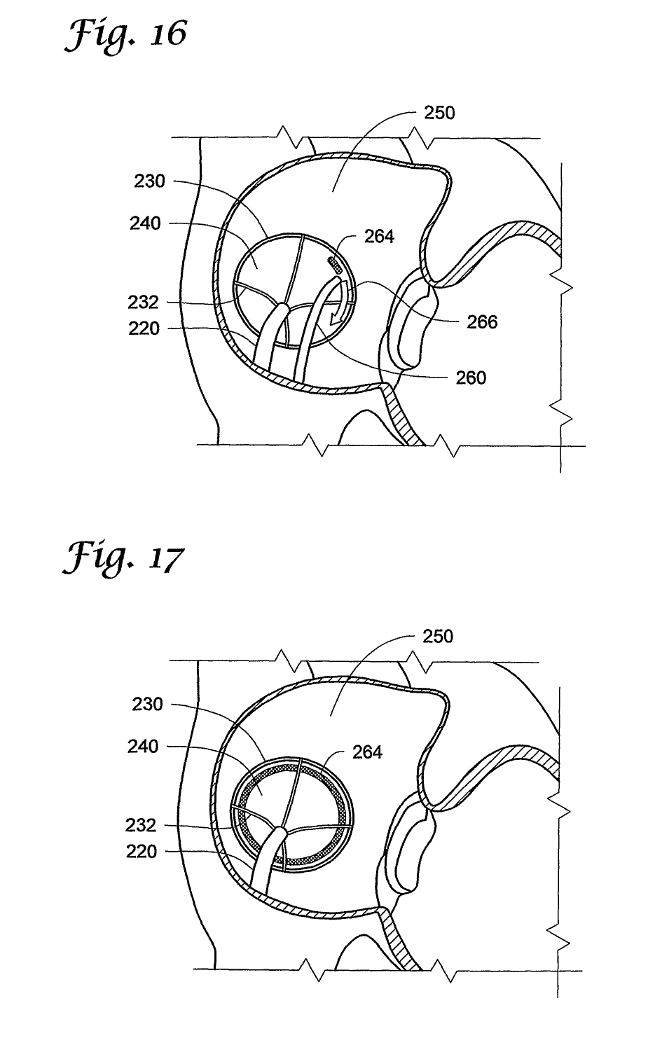

Although not required, it may be preferred that the patch 240 be attached to the atrial septum in an area 264 that extends about the perimeter of the patch as seen in FIGS. 16 & 17. It may be preferred that the attachment catheter be advanced sequentially about the perimeter of patch 240 as indicated by arrow 266 in FIG. 16 until the patch 240 is sufficiently attached.

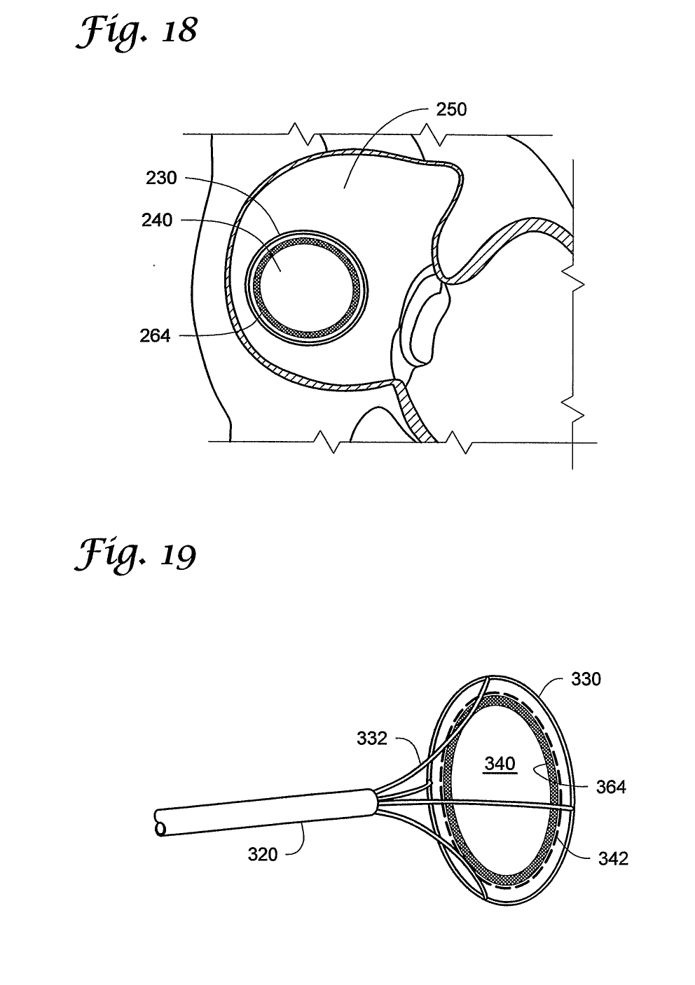

With the patch 240 attached to the epicardial surface/wall (atrial septum) of the right atrium, the struts 232 may be detached from the perimeter support 230 of the deployment scaffold and withdrawn back into the delivery sheath 220. The delivery sheath 220 may then be withdrawn from the right atrium 250, leaving the patch 240 attached at welded area 264 as depicted in FIG. 18.

In one potential alternative to the use of a separate catheter 260 to deliver energy to attach patch 240, the energy may potentially be delivered using the perimeter support 230 and/or struts 232 of the deployment scaffold.

In the embodiment depicted in FIG. 18, the perimeter support 230 remains attached to the patch 240. In some apparatus according to the present invention, it may be possible to remove the perimeter support from the patch after attachment to the atrial septum (or other internal body location). FIGS. 19-23 depict an example of one such embodiment and an exemplary method of using the apparatus. In other embodiments, perimeter support 230 may be constructed of degradable materials such that a rigid foreign body is no longer present after, e.g., several weeks. For example, the perimeter support may be constructed of a degradable bio-polymer, etc.

FIG. 19 is a perspective view of the closure apparatus which includes a delivery sheath 320 with a patch 340 deployed from its distal end. The patch 340 is held by a deployment scaffold that includes a perimeter support 330 and struts 332. The patch 340 is attached to, e.g., an atrial septum (not shown) within a welded area 364 as discussed herein. The patch 340 also includes a line of weakness 342 located outside of the welded area 364. The depicted line of weakness 342 may be in the form of a series of perforations formed in the patch 340. The line of weakness provides a path along which the patch 340 will preferentially separate when placed in tension as discussed herein.

It should be understood that other lines of weakness may be provided in place of a series of perforations, e.g., a thinned area in the patch at which the patch preferentially separates when under tension, different materials, etc. In some embodiments, the line of weakness may include adhesives (e.g., hot-melt adhesives) that become activated upon the application of energy to the patch and/or the scaffolds used in connection with the patch. Such adhesives may assist in separating the patch and/or attaching it in a desired location. In addition, the line of weakness may simply be formed by a material that weakens (e.g., separates) in response to energy used to attach the patch.

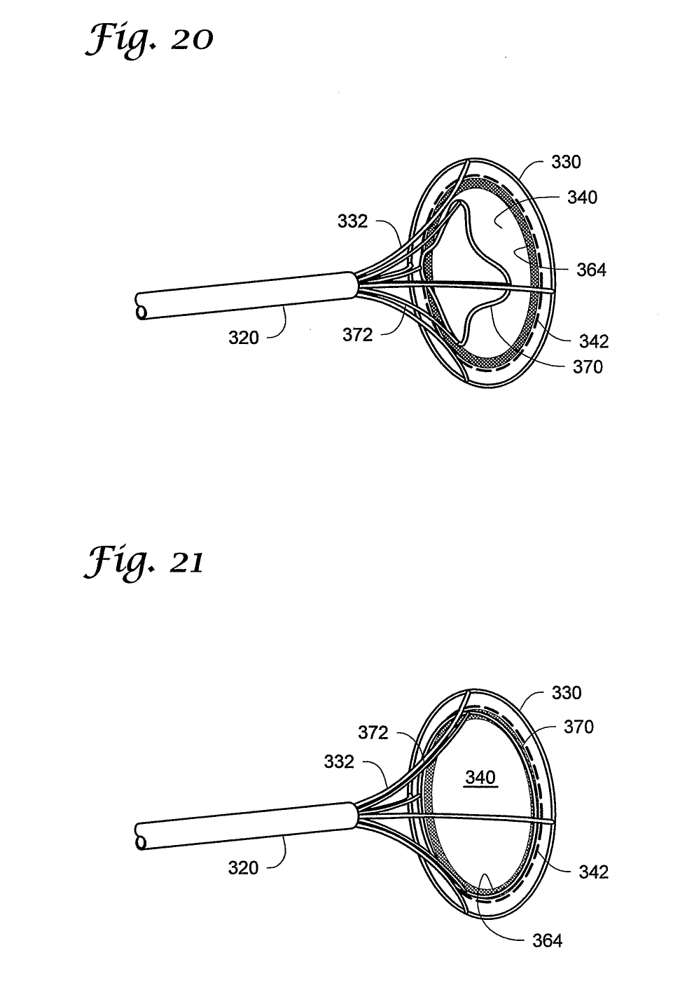

FIG. 20 depicts the deployment of a secondary scaffold from the delivery sheath 320 that, in the depicted embodiment, includes a perimeter support 370 and struts 372 in a construction somewhat similar to the deployment scaffold. In the depicted embodiment, the secondary scaffold is also capable of moving from a collapsed delivery configuration in which the secondary scaffold can be delivered through a lumen in the delivery sheath 320. Although the secondary scaffold is delivered using the same delivery sheath 320 in the depicted embodiment, the secondary scaffold could alternatively be delivered using a separate catheter or other device.

After advancement out of the delivery sheath 320, the secondary scaffold preferably expands to a deployment configuration as seen in FIG. 21. It may be preferred that the secondary scaffold be constructed of, e.g., shape memory materials to assist in the changes between the delivery configuration and the deployment configuration. Once deployed, the perimeter support 370 is preferably advanced towards the patch 340 such that the perimeter support 370 contacts the major surface of the patch 340 that faces the distal end of the delivery sheath 320. While the secondary scaffold 370 is depicted as circular in its deployed configuration to apply pressure at or near the line of weakness, in other embodiments the secondary scaffold may include cross-members and/or an energizable membrane that may preferably be capable of applying more uniform pressure across the patch 340. Such a design may also be capable of providing more uniform energy delivery over the surface of the patch.

With the perimeter support 370 of the secondary scaffold resting against the patch 340 and the perimeter support 330 of the deployment scaffold attached to the patch 340 outside of the perimeter support 370 of the secondary scaffold, tension can be applied across the line of weakness 342 to separate the patch 340 along the line of weakness 342. The tension may preferably be applied by holding secondary scaffold stationary against the patch 340 while drawing the perimeter support 330 back towards the delivery sheath 320.

In some instances, the secondary scaffold may be used to deliver energy to attach patch 340 at the desired location. The energy may potentially be delivered using the perimeter support 370 and/or struts 372 of the deployment scaffold.

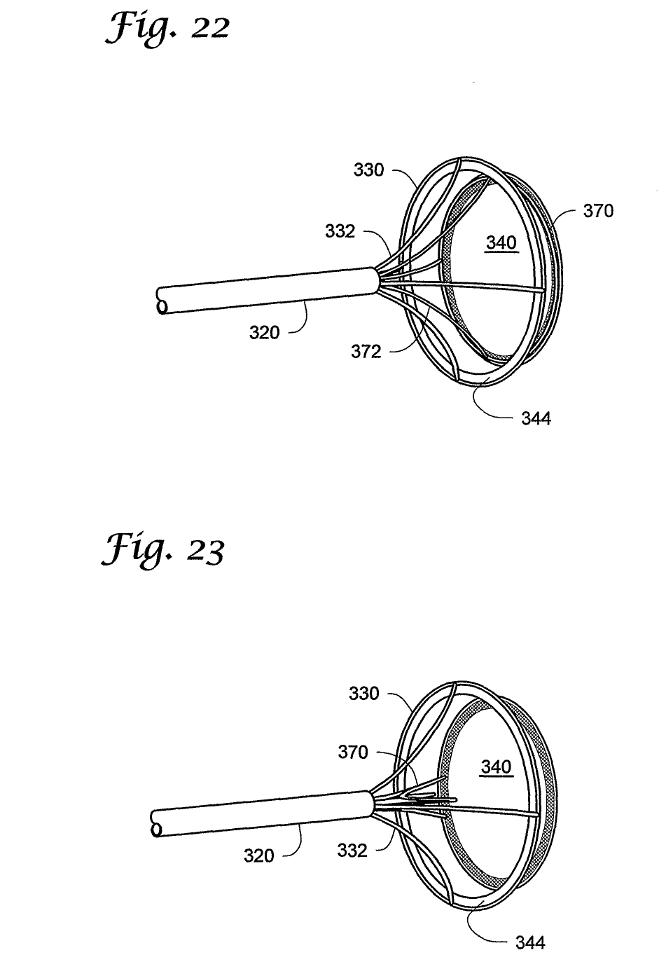

FIG. 22 depicts the patch 340 after separation along the line of weakness 342 with a portion 344 of the patch 340 located outside of the line of weakness 342 being removed along with the perimeter support 330. At the same time, the perimeter support 370 may preferably remain in contact with the portion of the patch 340 that remains attached to the atrial septum to assist avoiding detachment of the patch 340 from the atrial septum during separation of the patch 340 along the line of weakness 342.

After the patch 340 has been separated along the line of separation 342, the perimeter support 370 and struts 372 of the secondary scaffold may preferably be withdrawn in the proximal direction such that the secondary scaffold moves back into the delivery sheath 320 as depicted in, e.g., FIG. 23. Following withdrawal of the secondary scaffold into the delivery sheath 320, the perimeter support 330 and struts 332 of the deployment scaffold (and attached portion 344 of the patch 340) may also be withdrawn into the delivery sheath 320. Alternatively, the order of removal could be reversed, i.e., the deployment scaffold may be withdrawn into the delivery sheath first, followed by withdrawal of the secondary scaffold. In another alternative, the two scaffolds could potentially be withdrawn simultaneously (after attachment of the patch 340 and separation of the patch 340 along the line of weakness 342).

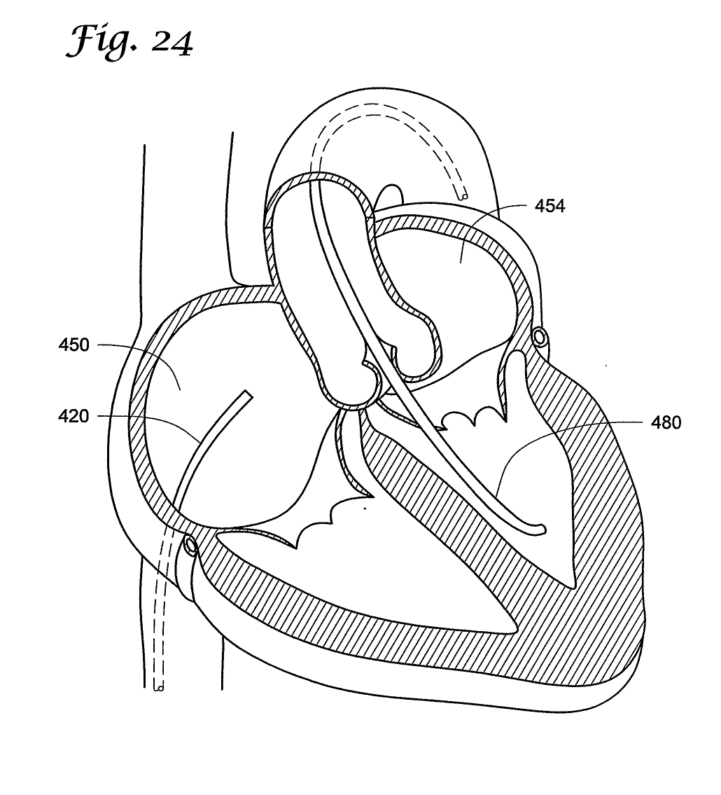

FIGS. 24-26 depict another closure apparatus and method according to the present invention. Unlike the apparatus and methods discussed above in connection with FIGS. 1-23 in which devices are deployed into only the right atrium, the apparatus and methods in FIGS. 24-26 involve temporarily deploying a pair of opposing devices, one into the right atrium 450 and one into the left atrium 454.

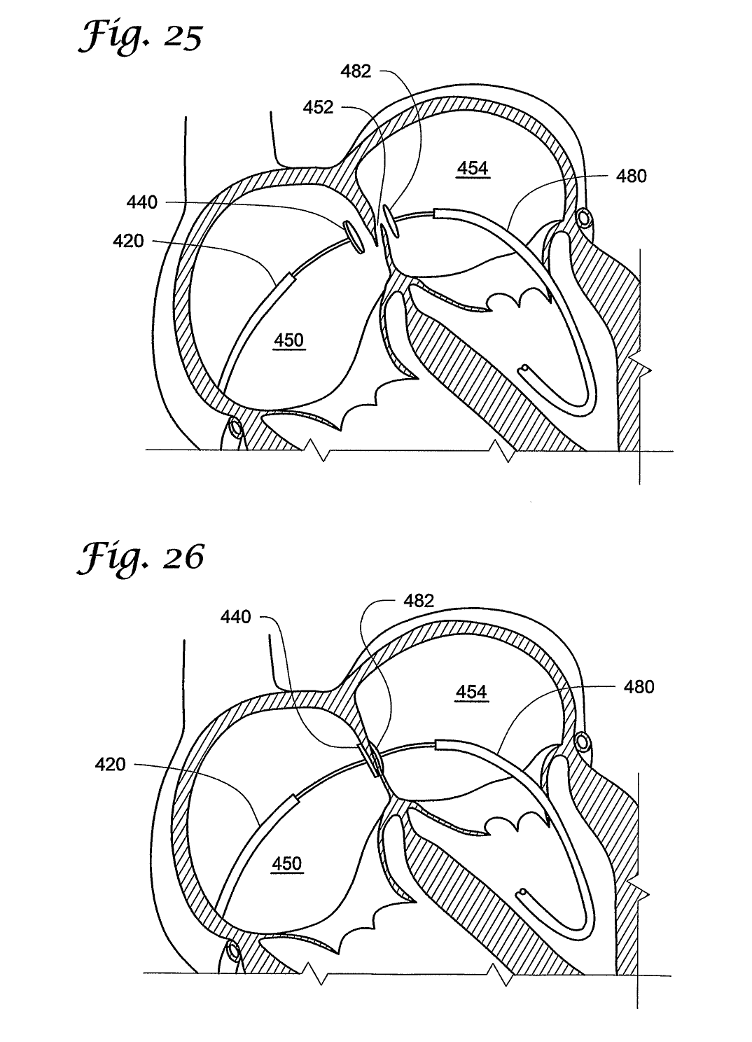

The apparatus and methods of FIGS. 24-26 rely on the use of energy (e.g., radio frequency (RF), thermal, etc.) to weld or fuse tissue together in much the same manner as discussed above in connection with the welding of a patch to close an opening. The apparatus and methods of FIGS. 24-26 could be used to close openings (such as patent foramen ovales in the atrial septum between the right and left atriums) in which tissue flaps overlap each other. Fusion or attachment of the opposing flaps surrounding opening 452 (see FIG. 25) between the right atrium 450 and the left atrium 454 may be used to close the opening 452.

The apparatus and method of FIGS. 24-26 include a first delivery sheath 420 deployed into the right atrium 450 and a second delivery sheath 480 deployed into the left atrium 454 using a retrograde aortic approach as depicted in FIG. 24. Both delivery sheaths 420 and 480 may preferably be delivered to the selected location percutaneously, although other methods of delivery could be used.

With the delivery sheaths 420 and 480 in position as seen in FIG. 25, a first component 440 may be deployed into the right atrium 450 from the first delivery sheath 420 and a second component 482 may be deployed into the left atrium 454 from the second delivery sheath 480. It may be preferred that the first component 440 and the second component 482 be magnetically attracted to each other to assist in aligning them across the opening 452, although other alignment methods could be used in place of magnetic attraction. For example, one or both of components 440 and 482 could be electromagnets, permanent magnets, etc. If in the form of electromagnets, magnetic attraction would be present when the electromagnet is activated. The magnetic attraction could provide mechanical pressure for tissue or patch welding in addition to aligning the components.

With the components 440 and 482 in alignment across the opening 452, they may preferably be advanced to compress the tissue located between them as depicted in FIG. 26 thereby preferably sealing the defect. With the tissue of opening 452 in position between components 440 and 482, energy is preferably emitted from at least one of the components and directed into the tissue located between them. It may be preferred that the energy be emitted from the first component 440 located in the right atrium, while the second component 482 is located opposite the first component 440. The second component may act as, e.g., an energy absorber, heat sink, etc. In one embodiment, bipolar RF energy could be delivered between the components, preferably reducing the risk of energy disruption of adjacent tissues and potentially improving localized energy delivery.

Although not depicted, the component 440 in the right atrium may be used to apply a patch that is attached to the internal body site during closure of the opening 452, with the patch remaining in position after the first component 440 and the second component 482 have been removed. Such a patch may be constructed similar to the patches discussed above and may include, e.g., tissue solder materials, etc.

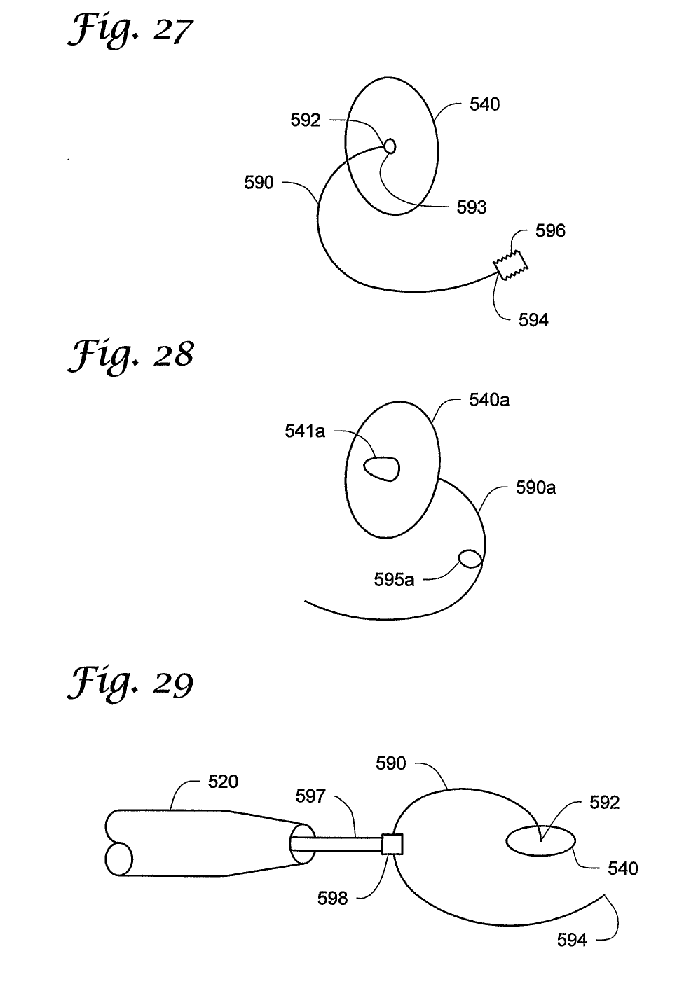

FIGS. 27-30 depict another closure apparatus and method according to the present invention. The apparatus and methods depicted in FIGS. 27-30 preferably involve the use of resilient forces to at least temporarily retain a patch in a selected position. The apparatus and methods of FIGS. 27-30 could be used to close openings (such as patent foramen ovales in the atrial septum between the right and left atriums).

Referring to FIG. 27, the closure apparatus itself may include a patch 540 and a clamping arm 590 attached thereto. The patch 540 may be collapsible as described in other embodiments herein and may or may not include perimeter supports, struts and other support structures designed to hold the patch in a desired shape. The patch 540 may also be constructed of the various materials described for patches herein.

The clamping arm 590 may preferably be in the form of a resilient curved member with a first end 592 attached to the patch 540 (or its support structure) and a second end 594. The clamping arm 590 is preferably manufactured with a shape and of materials such that deformation of the clamping arm 590 can generate a spring force. It may be preferred that the spring forces be generated when, for example, the first end 592 and the second end 594 of the clamping arm 590 are moved away from each other.

An optional feature depicted in connection with FIG. 27 is that the closure apparatus may include a connector 593 between clamping arm 590 and patch 540 (or its support structure) that allows for one or more degrees of freedom between the clamping arm 590 and the patch 540. For example, the clamping arm 590 may be connected to the patch 540 using a ball-socket joint, hinge, etc. such that the patch 540 can rotate about one or more axes relative to the clamping aim 590. Such a connection may allow the patch 540 to more firmly seat against tissue when the closure device is deployed within a patient.

The closure apparatus may also include a tissue anchor 596 at the second end 594 of the clamping arm 590. The tissue anchor may be used to secure the second end 594 of the clamping arm 590 at a selected internal body location. The tissue anchor 596 may take any suitable form, e.g., a threaded body, an expandable body, a barbed member, etc. that is capable of securing the second end 594 of the clamping arm 590 to the tissue found at a selected location.

As seen in FIG. 28, the clamping arm 590a may include, e.g., a coil 595a to further enhance the resilient forces generated upon deformation of the clamping arm 590a.

Another optional feature depicted in FIG. 28 that may be included in the closure apparatus of the present invention is the plug 541 a attached to the patch 540a. The plug 541a may be sized and positioned on the patch 540a such that plug 541 a may be inserted into an opening to be sealed, with the patch 540a surrounding the plug 541a. The plug 541 a may be constructed of any suitable material, e.g., a mesh, fabric, solid body, porous body, etc.).

In FIG. 29, the closure apparatus of FIG. 27 is depicted after advancement out of a delivery sheath 520. The closure apparatus is attached to an advancement member 597 that extends through the delivery sheath 520. The advancement member 597 may take the form of, e.g., a wire, a tube, or other elongated body that can be used to advance or retract the closure apparatus relative to the delivery sheath 520.

The advancement member 597 may preferably be releasably attached to the clamping arm 590 of the closure device using a connector 598. The connector 598 may preferably provide a hinged or jointed connection that provides one or more degrees of freedom between the advancement member 597 and the clamping arm 590. To provide the desired movement, the connector 598 may be in the form of a hinge, a ball-socket joint, etc.

It may be preferred that the point at which the advancement member 597 attaches to the clamping arm 590 at a location between the first end 592 and the second end 594 of the clamping arm 590. Alternatively, the advancement member 597 may be attached to the closure apparatus at the junction of the clamping arm 590 and the patch 540 (and/or its support structure).

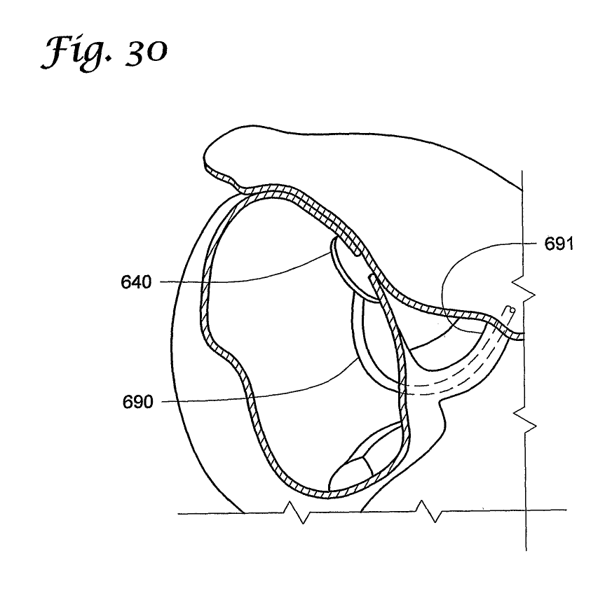

FIG. 30 depicts one embodiment of a closure apparatus of FIGS. 27-29 as deployed within a patient to close a PFO. The patch 640 is in position over a PFO while the clamping arm 690 extends into the coronary sinus 691 to provide the clamping force needed to retain the patch 640 in the selected position.

Deployment of the closure apparatus may preferably be achieved by advancing a delivery sheath (or catheter) using femoral or subclavian access into the coronary sinus 691. When in the coronary sinus 691, the second end of the clamping arm 690 would be advanced out of the delivery sheath into a selected location. It may be preferred that the second end of the clamping arm 690 be located proximate the junction between the coronary sinus 691 and the great cardiac vein (the vein of Marshall, and commonly the first posterolateral vein). From various studies, this measurement is typically between 2.5 to 4 cm, averaging about 3 cm.

As the clamping arm 690 moves out of the delivery sheath, the distal end of the sheath itself may preferably be withdrawn into the right atrium (moving up and posteriorly) to deploy the patch 640 in the selected location over a PFO.

Once in position, the patch 640 may be attached to the tissue surrounding the PFO by any suitable technique(s) as discussed herein, e.g., tissue welding, adhesives, etc.

In some embodiments, the clamping arm 690 may be used to hold the patch 640 in position only temporarily. The clamping arm 690 may, for example, be made of resorbable material. Alternatively, the clamping arm 690 may be removable such that, after an appropriate period of time, the clamping arm 690 may be retrieved from the deployment site, leaving the patch 640 in position. Removal may be accomplished by using a snare and sheath/catheter or by any other suitable technique.

The clamping arms in closure devices such as those depicted in FIGS. 27-30 take advantage of certain anatomical features to retain the patch in the selected location, e.g., the average distance between the coronary sinus (CS) ostium and the foramen ovale. In dissections of 47 hearts, the distance from the midpoint of the coronary sinus (i.e., the midpoint between the ostium to the junction with the great cardiac vein) to the patent foramen ovale or superior margin of the fossa ovalis was 9 millimeters (mm)±6 mm. This is different from a circumferential measurement using a sector starting from the midpoint of the coronary sinus to the patent foramen ovale. Regarding the planar orientation of the coronary sinus and foramen ovale, the foramen ovale and superior limbus were always in a posterior coronal plane in comparison to the coronary sinus with the plane of the IVC and eustachian ridge being in between these two planes in the examined hearts.

Another relationship discovered in this analysis was the angle between the coronary sinus and the patent foramen ovale. When measured relative to a line that was drawn perpendicular to the long axis of the heart into the coronary sinus, the angle to the PFO with a posterior and upward declination was 40 degrees±4 degrees. This limited angular variability may provide the basis for the design of a clamping arm that can provide the proper amount of force to hold the patch in position as discussed herein.

Other variations that may not be specifically discussed above may include, e.g., the use of magnets on the clamping arm and/or patch to generate additional force to hold the patch in a selected location. Another variation is that the size of a plug (if any) used in conjunction with the patch may be larger than in the embodiment depicted in FIG. 28. In some embodiments, the patch itself may be optional, with only a plug being located at the end of the clamping arm and closing off an opening such as a PFO. Still another variation may involve the addition of a lumen into the clamping device such that patency of the coronary sinus can be maintained and/or other devices may be passed into or through the coronary sinus for alternative therapies (e.g., the placement of leads, etc.)

The materials used to construct the various closure apparatus of the present invention may preferably be those materials suitable for use in medical devices, e.g., metals, polymers, composite materials, etc. In addition, it may be preferred that the apparatus be adapted for percutaneous delivery.

As used herein and in the appended claims, the singular forms “a,” “and,” and “the” include plural references unless explicitly limited to the singular form or the context clearly dictates otherwise.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A percutaneous closure apparatus comprising:
a delivery sheath comprising a proximal end and a distal end, wherein one or more lumens extend from the proximal end to the distal end of the delivery sheath;
a collapsible patch comprising first and second major surfaces, wherein the patch is capable of collapsing into a delivery configuration amenable for delivery to an internal body location through one lumen of the one or more lumens of the delivery sheath, and wherein the patch is capable of moving from the delivery configuration into a deployment configuration in which the first and second major surfaces are generally flat when the patch is located outside of the one lumen of the one or more lumens;
a deployment scaffold attached to the patch, wherein the deployment scaffold is capable of collapsing into a delivery configuration amenable for delivery to an internal body location through the one lumen of the one or more lumens of the delivery sheath, and wherein the deployment scaffold is capable of moving from the delivery configuration into a deployment configuration in which the deployment scaffold supports the patch in its deployment configuration when the deployment scaffold and the patch are located outside of the one lumen of the one or more lumens; and
a plurality of struts attached to the deployment scaffold, wherein each strut extends from the deployment scaffold to the proximal end of the delivery sheath, and wherein the plurality of struts can move proximally and distally within the one lumen of the one or more lumens of the delivery sheath to move the patch and deployment scaffold within the delivery sheath.

2. An apparatus according to claim 1, wherein the patch comprises a degradable biopolymer.

3. An apparatus according to claim 1, wherein the patch comprises a synthetic membrane.

4. An apparatus according to claim 1, wherein the patch comprises a nonwoven porous matrix.

5. An apparatus according to claim 4, wherein the patch further comprises one or more cell recruitment factors attached to or located within the matrix.

6. An apparatus according to claim 1, wherein, when the patch is in its deployment configuration, the first major surface of the patch faces the distal end of the delivery sheath and the second major surface of the patch faces away from the distal end of the delivery sheath, and further wherein the second major surface comprises a tissue welding solder.

7. An apparatus according to claim 1, wherein the deployment scaffold comprises a perimeter support attached to the patch, wherein the perimeter support extends about a perimeter of the patch.

8. An apparatus according to claim 7, wherein the perimeter support comprises a continuous member forming a closed figure.

9. An apparatus according to claim 7, wherein the perimeter support comprises two or more members attached to the patch.

10. An apparatus according to claim 7, wherein the patch comprises a line of weakness formed therein, the line of weakness located inside of the perimeter support.

11. An apparatus according to claim 10, wherein the line of weakness comprises a row of perforations in the patch.

12. An apparatus according to claim 1, wherein the apparatus further comprises:
a secondary scaffold, wherein the secondary scaffold is capable of collapsing into a delivery configuration amenable for delivery to the internal body location through one lumen of the one or more lumens of the delivery sheath, and wherein the secondary scaffold is capable of moving from the delivery configuration into a deployment configuration when the secondary scaffold is located outside of the one lumen of the one or more lumens; and
a plurality of struts attached to the secondary scaffold, wherein each strut extends from the secondary scaffold to the proximal end of the delivery sheath, and wherein the plurality of struts can move proximally and distally within the one lumen of the one or more lumens of the delivery sheath to move the secondary scaffold within the delivery sheath.

13. An apparatus according to claim 12, wherein the secondary scaffold is capable of being advanced distally against the patch and away from the distal end of the delivery sheath such that the secondary scaffold is located within the deployment scaffold on the first major surface of the patch.

14. An apparatus according to claim 1, wherein the deployment scaffold comprises shape memory material.

15. An apparatus according to claim 1, wherein the struts attached to the deployment scaffold comprise are magnetically attached to the deployment scaffold.

16. An apparatus according to claim 1, wherein the struts attached to the deployment scaffold comprise interlocking mechanical connections with the deployment scaffold.

17. A method of closing a patent foramen ovale, the method comprising:
positioning a distal end of a delivery sheath into the right atrium of a subject;
advancing a closure apparatus out of a lumen of the delivery sheath and into the right atrium, wherein the closure apparatus does not enter the left atrium, and wherein the closure apparatus comprises:
a collapsible patch comprising first and second major surfaces, wherein the patch is capable of collapsing into a delivery configuration amenable for delivery to an internal body location through one lumen of the one or more lumens of the delivery sheath, and wherein the patch is capable of moving from the delivery configuration into a deployment configuration in which the first and second major surfaces are generally flat when the patch is located outside of the one lumen of the one or more lumens;
a deployment scaffold attached to the patch, wherein the deployment scaffold is capable of collapsing into a delivery configuration amenable for delivery to an internal body location through the one lumen of the one or more lumens of the delivery sheath, and wherein the deployment scaffold is capable of moving from the delivery configuration into a deployment configuration in which the deployment scaffold supports the patch in its deployment configuration when the deployment scaffold and the patch are located outside of the one lumen of the one or more lumens; and
a plurality of struts attached to the deployment scaffold, wherein each strut extends from the deployment scaffold to the proximal end of the delivery sheath, and wherein the plurality of struts can move proximally and distally within the one lumen of the one or more lumens of the delivery sheath to move the patch and deployment scaffold within the delivery sheath;
positioning a second major surface of the patch over one or more perforations in the atrial septum, wherein blood flow through the one or more perforations is inhibited by the patch; and
attaching the patch to the atrial septum by directing RF energy towards the atrial septum through a first major surface of the patch, wherein the RF energy passes through the second major surface of the patch and into the atrial septum, wherein the attaching comprises directing the RF energy to a series of discrete locations on the patch.

18. A method of closing a patent foramen ovale in an atrial septum, the method comprising:
positioning a distal end of a first delivery sheath in the right atrium of a subject;
positioning a distal end of a second delivery sheath in the left atrium of a subject:
advancing a first component out of a lumen of the first delivery sheath and into the right atrium, wherein the first component is located on one side of the atrial septum;
advancing a second component out of a lumen of the second delivery sheath and into the left atrium, wherein the second component is located opposite the first component on an opposing side of the atrial septum;
magnetically aligning the first component and the second component across the atrial septum;
delivering RF energy to the patent foramen ovale in the atrial septum to close the patent foramen ovale;
removing the second component and the second delivery sheath from the left atrium; and
removing the first component and the first delivery sheath from the right atrium.

19. A percutaneous closure apparatus comprising:
a delivery sheath comprising a proximal end and a distal end, wherein one or more lumens extend from the proximal end to the distal end of the delivery sheath;
a collapsible patch comprising first and second major surfaces, wherein the patch is capable of collapsing into a delivery configuration amenable for delivery to an internal body location through one lumen of the one or more lumens of the delivery sheath, and wherein the patch is capable of moving from the delivery configuration into a deployment configuration in which the first and second major surfaces are generally flat when the patch is located outside of the one lumen of the one or more lumens;
a deployment scaffold attached to the patch, wherein the deployment scaffold is capable of collapsing into a delivery configuration amenable for delivery to an internal body location through the one lumen of the one or more lumens of the delivery sheath, and wherein the deployment scaffold is capable of moving from the delivery configuration into a deployment configuration in which the deployment scaffold supports the patch in its deployment configuration when the deployment scaffold and the patch are located outside of the one lumen of the one or more lumens;
a clamping arm comprising a first end attached to the deployment scaffold, the clamping arm comprising a resilient curved member that generates a spring force when deformed that is sufficient to retain the collapsible patch in a selected location;
a tissue anchor attached at a second end of the clamping arm, wherein the second end of the clamping arm can be secured to a selected internal location; and
an advancement member extending through the delivery sheath, the advancement member attached to the clamping arm by a connector, wherein the connector provides one or more degrees of freedom in movement between the advancement member and the clamping arm.

20. A method of closing a patent foramen ovale, the method comprising:
providing a closure apparatus according to claim 19;
positioning the distal end of the delivery sheath into the right atrium of a subject;
advancing the closure apparatus out of a lumen of the delivery sheath and into the right atrium;
positioning the collapsible patch at a selected location over one or more perforations in the atrial septum, wherein blood flow through the one or more perforations is inhibited by the patch;
retaining the collapsible patch at the selected location by advancing the second end of the clamping arm into the coronary sinus and attaching the tissue anchor to tissue in the coronary sinus, wherein the clamping arm is partially deformed such that a spring force is generated between the first end and the second end of the clamping arm; and
directing RF energy towards the atrial septum towards the collapsible patch, wherein the RF energy passes through the patch and into the atrial septum, wherein the RF energy welds the patch to the atrial septum.
